SEQUENCE LISTING (1 sequence)

SEQ ID NO: 1 - protein, 733 aa, Geobacillus
TTSTGALGPVTPKDTIYQIVTDRFFDGDPSNNKPPGFDPTLFDDPDGNNQGNGKDLKLYQGGDFQGIIDKIPYLKNMGITAVWISAPYENRDTVIEDYQSDGSINRWTSFHGYHARNYFATNKHFGTMKDFIRLRDALHQNGIKLVIDFVSNHSSRWQNPTLNFAPEDGKLYEPDKDANGNYVFDANGEPADYNGDGKVENLLADPHNDVNGFFHGLGDRGNDTSRFGYRYKDLGSLADYSQENALVVEHLEKAAKFWKSKGIDGFRHDATLHMNPAFVKGFKDAIDSDAGGPVTHFGEFFIGRPDPKYDEYRTFPERTGVNNLDFEYFRAATNAFGNFSETMSSFGDMMIKTSNDYIYENQTVTFLDNHDVTRFRYIQPNDKPYHAALAVLMTSRGIPNIYYGTEQYLMPSDSSDIAGRMFMQTSTNFDENTTAYKVIQKLSNLRKNNEAIAYGTTEILYSTNDVLVFKRQFYDKQVIVAVNRQPDQTFTIPELDTTLPVGTYSDVLGGLLYGSSMSVNNVNGQNKISSFTLSGGEVNVWSYNPSLGTLTPRIGDVISTMGRPGNTVYIYGTGLGGSVTVKFGSTVATVVSNSDQMIEAIVPNTNPGIQNITVTKGSVTSDPFRYEVLSGDQVQVIFHVNATTNWGENIYVVGNIPELGSWDPNQSSEAMLNPNYPEWFLPVSVPKGATFEFKFIKKDNNGNVIWESRSNRVFTAPNSSTGTIDTPLYFWDN

(19) United States
(12) Patent Application Publication
(10) Pub. No.: US 2021/0403967 A1
(43) Pub. Date: Dec. 30, 2021

(54) DIETARY FIBER PRODUCTION USING A GLYCOSYL-TRANSFERASE

(71) Applicant: Archer Daniels Midland Company, Decatur, IL (US)

(72) Inventors: Wu-Li Bao, Champaign, IL (US); Leif Solheim, Decatur, IL (US)

(21) Appl. No.: 17/280,852

(22) PCT Filed: Sep. 27, 2019
(86) PCT No.: PCT/US2019/053582

§ 371 Date: Mar. 26, 2021

Related U.S. Application Data
(60) Provisional application No. 62/737,398, filed on Sep. 27, 2018.

(51) Int. Cl. C12P 19/18; A23L 29/30; A23L 33/21; A23L 33/125; C12N 9/10

(52) U.S. Cl. CPC C12P 19/18; A23L 29/35; C12R 2001/07; A23L 33/125; C12N 9/1048; A23L 33/21

(57) ABSTRACT

Methods are disclosed for the production of soluble dietary fiber from a starch, including, e.g., corn and wheat starch. The methods comprise adding an acid to a soluble starch, mixing and heating the starch to form a starch substrate, and placing the starch substrate in contact with a glycosyl-transferase, thus producing a soluble dietary fiber composition having a higher a higher soluble dietary content than a composition without the addition of the glycosyl-transferase.

This application incorporates by reference the contents of a 5.75 kb text file named “CP0167US00sequencelisting.txt,” created on Sep. 27, 2018, which is the sequence listing for this application.

### BACKGROUND

Soluble dietary fiber, also referred to as indigestible fiber, has many benefits to human health and is marketed and sold as a functional food ingredient. Humans and other mammals can hydrolyze digestible starch, but not indigestible fiber or soluble dietary fiber, which passes through a mammal's digestive track without being hydrolyzed. Conventionally, soluble dietary fiber is manufactured from starch by an acidic thermal reaction, which at the same time generates a byproduct effluent waste. During manufacturing, proper control of the chemical process reaction parameters is necessary to avoid color formation, reduced yield, or increased byproducts that lead to economic losses from increased cost of production.

There are numerous plant sources of starch that have been described so far (Wikipedia; https://en.wikipedia.org/wiki/Starch). In plants, starch acts as an energy storage source. In 2008, the worldwide production of starch from all plant sources exceeded 66 million tons with approximately 60% used for food purposes and the remaining 40% used in industrial applications (Wikipedia; https://en.wikipedia.org/wiki/Starch). Starch is a mixture of amylose and amylopectin compounds, which are polymers of glucose linked by linear α-1,4 and branched α-1,6 glucosidic bonds. Differences in the amylose and amylopectin composition in plant-derived starches contribute to the temperature at which starches gelatinize and to their solubility in alcohol and aqueous solutions. Starch is hydrolyzed in biological system by amylases to glucose, a carbohydrate that provides energy and metabolic intermediates to cells. Conventional processing of starch uses acid or enzyme treatment or a combination of both. The degree of hydrolysis of the products is determined by the reducing sugar method that measures reactivity of the anomeric carbon as the free dextrose equivalent and reports as in DE units. The product is characterized as to the size of the dextrins, such as Dextrin DE5, or Dextrin DE10.

An example of acid treatment to produce indigestible fiber is disclosed in U.S. Pat. No. 5,139,575. That patent discloses a process of preparing indigestible heteropolysaccharides which features dissolving starch decomposition products and at least one kind out of monosaccharides excluding glucose, homo-oligosaccharides excluding glucooligosaccharides, and heterooligosaccharides into water and to which an inorganic acid was added, then powdering and heating the powder in an anhydrous condition thereof. In disclosed embodiments, a mixture of starch hydrolysate and a saccharide are dissolved in water, adding any acid, and then dried in a spray dryer. The dried mixture is then placed in a vat and heated in an oven. The powder is then dissolved in water and neutralized with sodium hydroxide, decolorized with activated charcoal, the desalted with ion-exchanger resins and finally spray-dried to obtain a powder comprising an indigestible portion.

U.S. Pat. No. 5,620,873 discloses a process for preparing a dextrin containing a dietary fiber characterized by dissolving a pyrodextrin in water and causing α-amylase to act on the solution. The patent discloses adding an acid to a starch, predrying the mixture at about 100° to about 120° C. to a water content of about 5% and roasting the mixture at 150° to 220° C. for about 1 to about 5 hours to obtain a pyrodextrin. The pyrodextrin thus obtained is preferably about 1 to 10 in DE (dextrose equivalent). In preparing the pyrodextrin, monosaccharides or oligosaccharides can be added to the starch so that the resulting dextrin contains an increased proportion of indigestible dextrin. Usually 50 to 60 wt. % saccharide solution is added in an amount of up to about 10 wt. % based on the starch. The pyrodextrin is then dissolved in water to a concentration of 30 to 50 wt. % and neutralized to a pH of 5.5 to 6.5. Commercial α-amylase (derived from conventional fungi and bacterial source) is added to the solution in an amount of 0.05 to 0.2 wt. % based on the pyrodextrin, and the solution is maintained at a temperature of about 85° to about 100° C. for 30 minutes to 2 hours, permitting the enzyme to act on the dextrin, whereby the dextrin is enzymatically decomposed to α-limit dextrin. The temperature is thereafter elevated to 120° C. to terminate the activity of α-amylase. The patent states that the above treatment removes odor and undesirable taste from pyrodextrin without greatly increasing the low digestibility thereof, permitting the dextrin to remain sparingly digestible as contemplated.

U.S. Pub. No. 2014/0023748 discloses a method for producing rice cakes or noodles, including the step of heat-treating a dough containing maltotriosyl transferase thereby gelatinizing starch in the dough. Also disclosed is a method for producing indigestible saccharide, including a step of allowing maltotriosyl transferase to act on a saccharide.

U.S. Pat. No. 8,546,111 discloses a glycosyltransferase and the use thereof, wherein the glycosyltransferase catalyzes transglucosylation of maltotriose units under conditions which can be employed for the processing of foods or the like. Disclosed is a maltotriosyl transferase which acts on polysaccharides and oligosaccharides having (X-1,4 glucoside bonds, and has activity for transferring maltotriose units to saccharides, themaltotriosyl transferase acting on maltotetraose as substrate to give a ratio between the maltoheptaose production rate and maltotriose production rate of 9:1 to 10:0 at any substrate concentration ranging from 0.67 to 70% (W/v).

“Indigestible Fractions of Starch Hydrolysates and Their Determination Method,” J. Appl. Glycosi., Vol 0.49, No. 4, p. 479-485 (2002), discloses that several types of starch hydrolysates that have dextrose equivalents of 12-16 were compared in terms of structural analysis, in vitro digestibility via the Prosky method, and digestibility as estimated by measuring the glycemic index in humans.

U.S. Pat. No. 5,492,829 discloses a particular strain of Klebsiella oxytoca No. 19-1 isolated from soil which produces a cyclomaltoglucanotransferase enzyme capable of converting starch to α-cyclodextrin in very high proportion, nearly close to 100 percent, rather than other types of cyclodextrins.

U.S. Pat. No. 3,819,484 discloses a sweetener having some of the properties of dextrin while being practically free of reducing sugars is prepared by subjecting sucrose and dextrin to cyclodextrin-glycosyl-transferase in an aqueous medium. Depending on the ratio of sucrose and dextrin, the product obtained after destroying the enzyme and purifying the fermentation mixture may be as sweet as an equal weight of the sucrose used or primarily show the properties of dextrin solution. The patent states that the product is useful in preparing food in which either or both properties are desired and is more stable thermally and chemically than sweeteners containing reducing sugars.

U.S. Pat. No. 9,657,322 discloses application of α-glucanotransferases in methods for preparing dietary fibers, including prebiotic oligosaccharides, and to oligosaccharides obtainable thereby. The patent states that a method for producing a mixture of glucooligosaccharides having one or more consecutive (α1→6) glucosidic linkages and one or more consecutive (α1→4) glucosidic linkages, comprises contacting a poly- and/or oligosaccharide substrate comprising at least two (α1→4) linked D-glucose units with an α-glucanotransferase capable of cleaving (α1→4) glucosidic linkages and making new (α1→4) and (α1→6) glucosidic linkages.

U.S. Pat. No. 4,477,568 discloses a process for producing cyclodextrin from starch in which an aqueous solution of starch is subjected to the action of an active cyclodextrin glycosyltransferase and the reaction mixture containing cyclodextrin, starch degradation products and active enzyme is continuously subjected to an ultrafiltration process to effect passage of the formed cyclodextrin through the membrane, while retaining substantially all of the other starch degradation products and active enzyme, thus permitting more cyclodextrin to be formed in the retentate, which will then pass the membrane, collecting the aqueous solution of cyclodextrin and recovering the cyclodextrin.

U.S. Pat. No. 9,005,681 discloses a method of producing a starch gel-containing food, the method comprising the steps of: treating starch granules with an enzyme at a temperature of about 10° C. or higher and about 70° C. or lower to obtain an enzyme-treated starch; mixing a food material, the enzyme-treated starch and water to obtain a mixture; heating the mixture thereby gelatinizing the enzyme-treated starch in the mixture; and cooling the mixture containing the gelatinized enzyme-treated starch thereby gelling the starch to obtain a starch gel-containing food. The enzyme is selected from the group consisting of amyloglucosidase, isoamylase, α-glucosidase, α-amylase having a characteristic capable of improving a gel forming ability of a starch, and cyclodextrin glucanotransferase.

WO2017/046040 discloses a method of producing a branched α-glucan. The patent discloses a branched n-glucan comprising alternating α(1→4) and α(1→6) glucosidic linkages and having α(1→4,6) branching points, a food composition, and the use of an α-glucanotransferase enzyme for reducing the digestible carbohydrates of a starch containing food material.

There continues to be a need for efficient methods for producing products with high soluble dietary fiber content, including methods wherein corn or wheat is the feedstock plant source.

### BRIEF DESCRIPTION OF THE FIGURES

FIG. 1A illustrates the pH of one embodiment used to create a fiber of the present invention.

FIG. 1B illustrates the pH of another embodiment used to create a fiber of the present invention.

### SUMMARY OF THE INVENTION

Aspects of the invention are associated with the discovery of processes for producing products with high soluble dietary fiber content using glycosyl-transferase (also known as maltotriose transferase, or maltotriosyl transferase, or GT). In an aspect, a process comprises adding an acid to a soluble starch, mixing and heating the starch to form a starch substrate, and placing the starch substrate in contact with a glycosyl-transferase, thus producing a soluble dietary fiber composition having a higher soluble dietary content than a composition without the addition of the glycosyl-transferase.

In an aspect, a process comprises increasing soluble dietary fiber content of a soluble dextrin. The process comprises placing a soluble dextrin in contact with glycosyl-transferase, thus increasing the soluble dietary fiber content of the soluble dextrin.

The processes disclosed herein may be used as an alternative to or an improvement of conventional processes for the manufacturing of soluble dietary fiber. The processes disclosed herein may be used to produce soluble dietary fiber at lower temperature and/or at a neutral pH with higher yield and reduced losses to color and byproduct formation than processes that do not include adding glycosyl-transferase. Use of glycosyl-transferase as disclosed herein can help reduce the risk of unstable products, adverse products, or products that result in off specification color during heating.

These and other aspects and associated advantages, as well as a number of particular embodiments, will become apparent in the following Detailed Description.

### DETAILED DESCRIPTION

A process for preparing soluble dietary fiber comprises receiving dry starch, and adding an acid, such as hydrochloric acid, to the dry starch in an acidification step. The process further comprises mixing and heating the starch and acid in vessel, wherein the acid acts as a catalyst to rearrange or create new polysaccharide glucosidic bonds at a raised temperature (e.g., about 100-135° C.). Next, the admixture may be conveyed to another vessel, and roasted at a temperature greater than the heating step temperature (e.g., about 140-150° C. In an embodiment, the heating step may be for about 30-90 minutes, and the roasting step may be for a shorter period of time, e.g., about 0.5-1.0 minutes. Both the heating step and the roasting step allow acidic thermal reaction to occur. About 40-50% of starch is converted to soluble dietary fiber during the heating step, and about another 3-10% of starch is converted to soluble dietary fiber during the roasting step. Thus, of the soluble dietary fiber that is formed, about 75-94% is formed during the heating step, and about 6-25% is formed during the roasting step. The roasting step has a tendency to cause undesirable color formation, such as a browning. After the roasting step, the process may further comprise addition of water to the starch to form a slurry, followed by addition of amylase enzymes in a liquefaction and saccharification step to process and purify the soluble dietary fiber.

In an aspect, the processes disclosed herein provide production of soluble dietary fiber from corn and wheat, and other plants, such as rice, cassava, barley, sorghum bean and potato. Corn, wheat, and other plant feedstocks may be material derived from non-genetically modified organisms (“non-GMO”). In an embodiment, the processes disclosed herein may be used to produce soluble dietary fiber from different plant streams, e.g., different corn starch dextrin streams or from different wheat streams. In an embodiment, a process comprises placing a starch or dextrin substrate in contact with a glycosyl-transferase, thus producing a soluble dietary fiber composition having a higher soluble dietary content than a composition without the addition of the glycosyl-transferase. In an embodiment, the glycosyl-transferase is an enzyme protein from a microorganism belonging to the genus Geobacillus. In an embodiment, the Geobacillus is Geobacillus sp. APC9669 (deposited with the Patent Microorganisms Depository, NITE Biotechnology Development Center, 2-5-8, Kazusakamatari, Kisarazu-shi, Chiba, 292-0818, Japan) under accession number NITE BP-770). In an embodiment, the glycosyl-transferase is an enzyme protein of about 83 kD (SDS-PAGE) from a microorganism. In an embodiment, the glycosyl-transferase comprises the amino acid sequence shown in SEQ ID NO:1.

Without being bound by theory, it is believed that the enzyme glycosyl-transferase removes a maltotriose from the non-reducing end of the dextrin and places it on the same or another molecule of dextrin as the glycosidic side chain. As more side chains form from the repeated transferase reactions, the product will become more soluble and be less accessible to digestion by amylase enzymes. The end product that is produced by using the glycosyl-transferase is desirable for use as a dietary fiber.

The glycosyl-transferase (also called maltotriosyl transferase) used in the examples herein was provided by Amano Enzyme Inc. (Nagoya-shi, Japan). U.S. Publication 2014/0004226, assigned to Amano Enzyme Inc., discloses a method for producing a maltotriosyl transferase. The publication discloses that the maltotriosyl transferase was isolated from a microorganism belonging to the genus Geobacillus sp. APC9669 (accession number NITE BP-770), and that its molecular weight is about 83,000 (SDS-PAGE). The publication also describes the amino acid sequence and the DNA sequence of the maltotriosyl transferase. The publication discloses that the enzyme acts on polysaccharides and oligosaccharides having α-1,4 glucoside bonds to transfer maltotriose units to oligo- and poly-saccharides. Regarding substrate specificity, the publication discloses that the maltotriosyl transferase acts on soluble starch, amylose, amylopectin, maltotetraose, maltopentaose, and maltohexaose, while it does not act on α-cyclodextrin, β-cyclodextrin, γ-cyclodextrin, maltotriose, and maltose.

In an aspect, a process for producing soluble dietary fiber comprises receiving a starch, e.g., a dry starch, adding an acid to the starch in an acidification step, mixing and heating the starch to form a starch substrate. The process comprises adding glycosyl-transferase (also known as maltotriose transferase, or maltotriosyl transferase, or GT) to the starch substrate to form a soluble dietary fiber composition having a higher soluble dietary content than a composition made in the same manner with the exception of the addition of the glycosyl-transferase. The acid used in the acidification step may comprise hydrochloric acid, e.g., dilute hydrochloric acid. In an embodiment, the combination of starch and acid has a pH in the range of about 1.0-3.0, and more preferably about 1.7.

In an embodiment, mixing and heating the starch is conducted at a temperature in the range of about 80°-135° C., preferably about 100-135° C.

In an embodiment, the glycosyl-transferase may be in the form of a powder. In an embodiment, the glycosyl-transferase may be in the form of a powder in the range of about 0.001-2.0% w/w of the starch substrate, more preferably about 0.018-1.2% w/w of the starch substrate, and even more preferably about 0.1% w/w of the starch substrate. In another embodiment, the glycosyl-transferase is in liquid form, preferably wherein the glycosyl-transferase is about 0.1% w/w of the starch substrate. Using the glycosyl-transferase in liquid form will typically provide easier handling than powder form. In addition, the enzyme reaction should occur in an aqueous buffer, so having the glycosyl-transferase in liquid form to begin with eliminates the step of having to convert the glycosyl-transferase from powder or solid form to liquid form.

In an embodiment, a buffer may be added so that the enzyme reaction between the starch substrate and the glycosyl-transferase may be conducted at a pH in the range of about 5.0-7.0, more preferably about 5.8-6.2, and even more preferably at about 6.0. In an embodiment, the buffer may be a phosphate buffer. In an embodiment, enzyme reaction may be conducted in a reactor, e.g., a rotisserie (or any agitated) reactor, at around 50-60° C., more preferably about 55° C. When a rotisserie reactor is used, it may be operated at about 40-60 revolutions per minute (rpm), more preferably 50 rpm.

In an embodiment, the process comprises adding an acid to a starch, mixing and heating the acidified starch to form a starch substrate and allowing acidic thermal reaction to occur, and roasting the starch substrate to allow further acidic thermal reaction to occur to form a roasted starch intermediate, and then adding a glycosyl-transferase to form a soluble dietary fiber composition having a higher soluble dietary content than a soluble dietary fiber composition without the addition of the glycosyl-transferase. The mixing and heating may be conducted in a dryer with a temperature in the range of about 100-135° C., for a predetermined period of time (e.g., 30-90 minutes). The roasting of the starch substrate may be conducted at a temperature in the range of about 140-180° C., preferably 140-150° C. for a predetermined period of time (e.g., about 0.5-1.0 minute) in a vessel separate from the vessel used for heating step.

Those skilled in the art will recognize that with the benefit of the present disclosure, an agitated reactor is preferable over a rotisserie reactor for the enzyme reaction between the glycosyl-transferase and the starch intermediate, particularly for large scale reactions. Enzyme reaction may be conducted about 5-40 hours, more preferably about 10-30 hours, and even more preferably about 20 hours.

In an aspect, a process comprises increasing dietary fiber content of a soluble dextrin. The process comprises placing the soluble dextrin in contact with glycosyl-transferase, such as in a reactor (e.g., a rotisserie or agitation reactor), thus increasing the dietary fiber content of the soluble dextrin. In an embodiment, the soluble dextrin has a dextrose equivalent (DE) of at least 5. In an embodiment, the process may comprise adding a buffer to the soluble dextrin prior to placing the soluble dextrin in contact with glycosyl-transferase. In an embodiment, the soluble dextrin derived from a dextrin source selected from the group consisting of corn and wheat, and other plants, such as rice, cassava, barley, sorghum bean and potato, and combinations thereof. While the examples below disclose production of soluble dietary fiber from corn and wheat feedstocks, those skilled in the art will recognize that with the benefit of the present disclosure, other plant feedstocks, including feedstocks derived from rice, cassava, barley, sorghum bean and potato may be used to produce soluble dietary fiber.

Examples below describe production of soluble dietary fiber using the enzyme glycosyl-transferase from: (1) dextrin DE5; (2) dextrin DE10; (3) 50% dietary fiber stream “A” (i.e., an intermediate of an acidic process from the steps of adding an acid to a dry starch, followed by mixing and heating of starch and acid at the same heating temperature and heating time period as previously discussed, and then roasting of the starch and acid at the same roasting temperature and roasting time period as previously discussed, referred to herein as “Fiber A”); and (4) 40% dietary fiber stream “B” (i.e., an intermediate of an acidic process from the steps of adding an acid to a dry starch, followed by mixing and heating of starch and acid at the same heating temperature and heating time period as previously discussed, referred to herein as “Fiber B”).

Production of dietary fiber from different corn dextrin streams.

Examples: Enzyme reactions were carried out with particular substrates in the concentrations listed in the tables. The reactions also contained a phosphate buffer at pH 6.0, and GT enzyme powder at 1% w/w of the substrate, which is incubated in a rotisserie reactor at 55° C. and 50 rpm for about 20 hours. The dietary fiber analytical results from the glycosyl-transferase reactions with different substrates are shown in the following tables (numbers are in g/kg), and “DS” means dry solids, with the % dry solids being dissolved in deionized water, wherein DP2+dextrose means maltose (DP2) and dextrose.

As shown in Table 1, starting with corn DE10 dextrin, up to 48% dietary fiber can be produced in the enzyme reaction as opposed to no dietary fiber produced where no enzyme is used.

TABLE 1
Production of dietary fiber from corn DE10 dextrin with GT.

| | Dietary | DP2 + | | Dietary |
|---|---|---|---|---|
| | fibers | dextrose | Total | fiber |
| Reactants | g/kg | g/kg | g/kg | % |
| DE10 dextrin 40% DS (−zyme) | 0 | 367.1 | 367.1 | 0 |
| DE10 dextrin 40% DS (+zyme) | 178.74 | 213 | 391.7 | 45.6 |
| DE10 dextrin 45% DS (−zyme) | 0 | 406.2 | 406.2 | 0 |
| DE10 dextrin 45% DS (+zyme) | 208.41 | 228.7 | 437.1 | 47.7 |

As shown in Table 2, starting with corn DE5 dextrin, about 35% dietary fiber can be produced in the enzyme reaction as opposed to a much lower dietary fiber produced where no enzyme is used.

TABLE 2
Production of dietary fiber from corn DE5 dextrin with GT.

| | Dietary | DP2 + | | Dietary |
|---|---|---|---|---|
| | Fiber | dextrose | Total | fiber |
| Reactants | g/kg | g/kg | g/kg | % |
| DE5 dextrin 25% DS (−zyme) | 4.0 | 248.1 | 252.1 | 1.6 |
| DE5 dextrin 25% DS (+zyme) | 91.1 | 168.2 | 259.3 | 35.1 |
| DE5 dextrin 35% DS (−zyme) | 4.1 | 326.1 | 330.2 | 1.2 |
| DE5 dextrin 35% DS (+zyme) | 114.0 | 218.1 | 332.1 | 34.3 |
| DE5 dextrin 45% DS (−zyme) | 4.4 | 441.1 | 445.5 | 1.0 |
| DE5 dextrin 45% DS (+zyme) | 126.3 | 316.4 | 442.7 | 28.5 |

As shown in Table 3, using Fiber A dextrin as a substrate, the enzyme can add about 10% more dietary fiber to the product as opposed to the reaction without using the enzyme. Fiber A is an intermediate of an acidic process wherein Fiber A is formed from the steps of adding an acid to dry starch, followed by heating and roasting, and allowing for a predetermined reaction time between the Fiber A dextrin and glycosyl-transferase (GT) of about 20 hours in a reactor.

TABLE 3
Production of dietary fiber from Fiber A dextrin with GT.

| | Dietary | Dextrose + | | Dietary |
|---|---|---|---|---|
| | Fiber | DP2 | Total | fiber |
| Reactants | g/kg | g/kg | g/kg | % |
| Fiber A 30% DS (−zyme) | 133.1 | 141.5 | 274.7 | 50.4 |
| Fiber A 30% DS (+zyme) | 157.0 | 115.0 | 272.0 | 60.0 |
| Fiber A 40% DS (−zyme) | 178.1 | 190.6 | 368.7 | 50.2 |
| Fiber A 40% DS (+zyme) | 217.0 | 158.4 | 375.4 | 60.1 |
| Fiber A 50% DS (−zyme) | 222.0 | 237.3 | 459.4 | 50.3 |
| Fiber A 50% DS (+zyme) | 269.4 | 198.8 | 468.2 | 59.8 |

As shown in Table 4, from Fiber A and corn DE10 dextrin with GT can boost dietary fiber about 60%.

TABLE 4
Production of dietary fiber from Fiber
A and corn DE10 dextrin with GT.

| | Dietary | DP2 + | | Dietary |
|---|---|---|---|---|
| Reactants (40% DS) | fibers | dextrose | total | fiber |
| (all with GT) | g/kg | g/kg | g/kg | % |
| Fiber A 70% + 30% DE10 | 214.64 | 160.8 | 375.4 | 57.2 |
| Fiber A 80% + 20% DE10 | 208.96 | 157.6 | 366.5 | 57 |
| Fiber A 90% + 10% DE10 | 239.84 | 154.2 | 394 | 60.9 |
| Fiber A 100% | 237.35 | 145.7 | 383.1 | 62 |

As shown in Table 5, from an intermediate product (Fiber B), treatment with GT increases the dietary fiber by over 25% at 30% dry solid mixture and by over 30% at 40% dry solid mixture in 24 hrs, as opposed to the process wherein no GT was used. Fiber B is an intermediate of an acidic process wherein Fiber B is formed from the steps of acidification of dry starch, followed by mixing and heating as previously discussed. The use of enzyme GT provides increased production of dietary fiber from intermediate products while also eliminating or reducing adverse effects in a process, e.g., color formation, because the roasting step at a temperature of about 140-180° C. or higher is eliminated.

TABLE 5
Production of more dietary fiber from intermediate
product Fiber B dextrin with GT.

| | Dietary | DP2 + | | Dietary |
|---|---|---|---|---|
| Reagents | Fibers | dextrose | total | Fiber % |
| Fiber B (30% DS) + GT | 162.26 | 131.21 | 293.47 | 59.7 |
| Fiber B (40% DS) + GT | 231.40 | 166.66 | 398.06 | 62.3 |
| Fiber B No GT | 406.47 | 562.81 | 969.28 | 47.7 |

As shown in Table 6, the fiber content from wheat dextrin WW82 is 18.2% without use of the enzyme GT, and the fiber content increases to 47.3% from wheat dextrin WW82 when the enzyme GT is used; the fiber content from wheat dextrin WC9526 is 46.2% without use of the enzyme GT, and the fiber content increases to 59.2% from wheat dextrin WC9526 when the enzyme GT is used; and the fiber content from corn CR15 is 0.8% without use of the enzyme GT, and the fiber content increases to 44.2% from corn CR15 when the enzyme GT is used.

TABLE 6
Production of dietary fiber from wheat dextrin WW82,
wheat dextrin WC9524, and corn DE15 dextrin with GT.

| | | Dietary | DP2 + | Dietary |
|---|---|---|---|---|
| | GT | fiber | dextrose | fiber |
| Dextrin | enzyme | g/kg | g/kg | % |
| WC9524 | Yes | 205.2 | 141.7 | 59.2 |
| WC9524 | no | 163.8 | 190.9 | 46.2 |
| WW82 | yes | 174.1 | 193.6 | 47.3 |
| WW82 | no | 64.5 | 289.7 | 18.2 |
| Corn CR15 | yes | 139.7 | 176.3 | 44.2 |
| Corn CR15 | no | 2.8 | 354.7 | 0.8 |

The GT enzyme can further be immobilized on a material so that the enzyme can be reused many times. The immobilized enzyme can also be packed into a column and the reaction substrate can pass through the immobilized enzyme resin. Those skilled in the art will recognize that the with the benefit of this disclosure, the immobilization of the GT enzyme may reduce costs and simplify downstream processing.

The processes disclosed herein provide a number of benefits, including but not limited to: (1) use of a glycosyl-transferase enzyme on corn starch process product dextrins DE5 and DE10 to produce soluble dietary fiber; (2) use of a glycosyl-transferase enzyme specifically on corn starch dietary fiber process product to increase fiber content; (3) use of a use of a glycosyl-transferase enzyme specifically on the combination of corn starch dextrins and a fiber stream to increase fiber content and production; (4) use of a glycosyl-transferase enzyme on corn starch dietary fiber process intermediates to improve the process, including reducing color and by-products); (5) use of a glycosyl-transferase enzyme specifically on wheat starch dextrin to produce soluble dietary fiber; and (6) immobilization of a glycosyl-transferase enzyme for the soluble dietary fiber production process.

As can be seen from the above results, significant increased yields of soluble dietary fiber can be produced under the disclosed reaction conditions and with the described reaction mixture components. It is expected that process optimization, based on the teachings herein, can be conducted to increase yields of soluble dietary fiber according to the synthesis methods and overall teachings set forth in the present disclosure.

Fiber was produced from a starch substrate substantially as described herein. The amount of dry solids (DS) of the starch substrate was varied from 30% to 60% and the viscosity of the starch substrate was determined at 60° C. The results are shown in Table 7.

TABLE 7
Effect of Substrate Dosing on Viscosity in Centipoise (cps).

| | | 30% DS | 40% DS | 50% DS | 60% DS |
|---|---|---|---|---|---|
| 3 | rpm | out of range | 115 | 1390 | 6239 |
| 6 | rpm | 29 | 67 | 726 | 3579 |
| 15 | rpm | 21 | 45 | 384 | 1928 |
| 30 | rpm | 11 | 38 | 245 | 1280 |
| 60 | rpm | 10 | 31 | 178 | 907 |

| | | | | |
|---|---|---|---|---|
| Spindle # | 18 | 18 | 27 | 34 |

The viscosity obtained for the starch substrate over 40% DS will have an impact on mixing which may lead to a variability in the fiber composition after the reaction with the enzyme. Being able to run the reaction on the starch substrate at a DS content of more than 30% means that less evaporation is needed which means the fiber may be exposed to less heat and have a better color. In various embodiments, the solids content of the present invention may be between about 30-60%, between about 35-55%, or between about 40-50%.

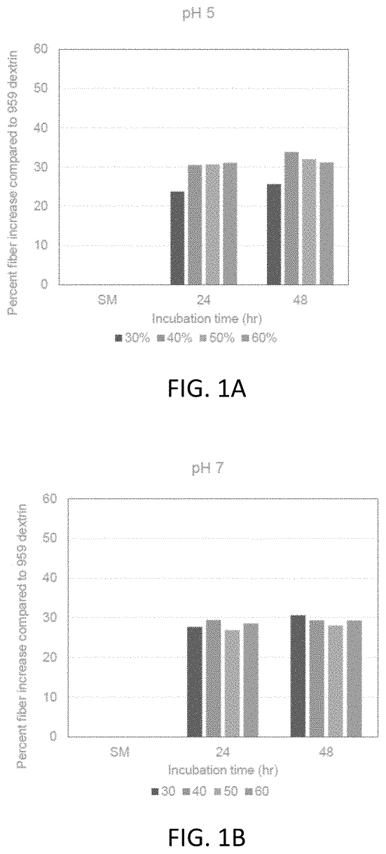

The effect of dry solids loading (from 30% to 60% DS) on the glycosyl transferase reaction from the starch substrate to fiber was also determined at pH 5 and pH 7. A dextrin starch substrate was treated with glycosyl transferase as a 1% dosage with respect to dry solids at pH 5 and pH 7 and incubated at 60° C. for 48 hours. Samples were taken at 24 hours and 48 hours. All samples were treated with 0.1% w/w glucoamylase and the fiber content was analyzed by HPLC. As shown in FIGS. 1A and 1B, the glycosyl transferase reaction worked at pH 5 and pH 7 without any added buffer. In various embodiments, the pH may between about 4 and 8, between about 4.5 and 7.5, between about 4.5 and 7, between about 4.5 and 6.5, between about 4.5 and 6, between about 4.5 and 5.5, or about 5.

In accordance the techniques described above, the present invention provides valuable processes for production of soluble dietary fiber from corn or wheat feedstock, including non-GMO feedstock. The present invention provides valuable processes for production of soluble dietary fiber with significant increased yields from intermediates that are typically produced in conventional processes. The processes of the present invention are particularly useful in the production of soluble dietary fiber since they eliminate or reduce the need for downstream processing required in conventional processing. The methods disclosed herein may advantageously address shortcomings of conventional methods.

While the aspects described herein have been discussed with respect to specific examples including various modes of carrying out aspects of the disclosure, those skilled in the art will appreciate that various changes can be made to these processes in attaining these and other advantages, without departing from the scope of the present disclosure. As such, it should be understood that the features of the disclosure are susceptible to modifications and/or substitutions without departing from the scope of this disclosure. The specific embodiments illustrated and described herein are for illustrative purposes only, and not limiting of the invention set forth in the appended claims.

1. A process of producing a fiber from a starch, comprising:
adding an acid to a soluble starch;
mixing and heating the starch and acid to form a starch substrate; and
placing the starch substrate in contact with a glycosyl-transferase, thus
producing a soluble dietary fiber composition having a higher soluble dietary content than a composition without the addition of the glycosyl-transferase.

2. (canceled)

3. The process of claim 1, wherein the mixing and heating are conducted at a temperature in the range of about 80-135° C.

4. (canceled)

5. The process of claim 1, wherein the glycosyl-transferase is an enzyme protein from a microorganism belonging to the genus Geobacillus.

6-7. (canceled)

8. The process of claim 1, wherein the glycosyl-transferase comprises the amino acid sequence shown in SEQ ID NO:1.

9. The process of claim 1, wherein the glycosyl-transferase is in the form of a powder in the range of about 0.001-2.0% w/w of the starch substrate.

10-11. (canceled)

12. The process of claim 1, wherein the glycosyl-transferase is in the form of a liquid.

13. (canceled)

14. The process of claim 1, wherein the enzyme reaction of the glycosyl-transferase and the starch substrate occurs at a pH in the range of about 4.5-6.0.

15. (canceled)

16. The process of claim 1, further comprising addition of a buffer so that the enzyme reaction of the glycosyl-transferase and the starch substrate occurs at a pH in the range of about 5.0-7.0.

17-18. (canceled)

19. The process of claim 1, wherein the enzyme reaction of the glycosyl-transferase and the starch substrate is conducted in a reactor and at a temperature in the range of about 50-60° C. for about 5-40 hours, wherein the reactor is selected from the group consisting of a rotisserie reactor and an agitated reactor.

20. The process of claim 1, wherein the starch is from the group consisting of corn, wheat, rice, cassava, barley, sorghum bean and potato, and combinations thereof.

21. The process of claim 1, wherein a solids content of the starch substrate is between about 35-55%.

22. (canceled)

23. A process of increasing dietary fiber content, comprising:
adding an acid to a starch;
mixing and heating the acidified starch to form a starch substrate;
roasting the starch substrate;
allowing acidic thermal reaction to occur to form a roasted starch intermediate;
adding a glycosyl-transferase to the roasted starch intermediate to form a soluble dietary fiber composition having a higher soluble dietary content than a composition without the addition of the glycosyl-transferase.

24. The process of claim 23, wherein the mixing and heating is conducted at a temperature in the range of about 80-135° C. for a period of about 30-90 minutes.

25-27. (canceled)

28. The process of claim 23, wherein the glycosyl-transferase comprises the amino acid sequence shown in SEQ ID NO: 1.

29. The process of claim 23, wherein the starch is from the group consisting of corn, wheat, rice, cassava, barley, sorghum bean and potato, and combinations thereof.

30. A process of increasing soluble dietary fiber content of a soluble dextrin comprising:
placing a soluble dextrin in contact with a glycosyl-transferase, thus increasing the soluble dietary fiber content of the soluble dextrin.

31. The process of claim 30, wherein the soluble dextrin has a dextrose equivalent (DE) of at least 5.

32-33. (canceled)

34. The process of claim 30, wherein the glycosyl-transferase comprises the amino acid sequence shown in SEQ ID NO: 1.

35. The process of claim 30, wherein the soluble dextrin is a dextrin derived the group consisting of corn, wheat, rice, cassava, barley, sorghum bean and potato, and combinations thereof.

36. The process of claim 30, wherein a buffer is added to the soluble dextrin before placing the soluble dextrin in contact with a glycosyl-transferase.
